(19) United States
(12) Plant Patent Application Publication
(10) Pub. No.: US 2021/0251115 P1
(43) Pub. Date: Aug. 12, 2021

(54) Blueberry plant named 'FC14-062'

(50) ;

(71) Applicant: Fall Creek Farm and Nursery Inc., Lowell, OR (US)

(72) Inventor: Peter Stefan BOCHES, Hilo, HI (US)

(73) Assignee: Fall Creek Farm and Nursery Inc., Lowell, OR (US)

(21) Appl. No.: 17/166,536

(22) Filed: Feb. 3, 2021

Related U.S. Application Data
(60) Provisional application No. 62/995,598, filed on Feb. 6, 2020.

(51) Int. Cl. A01H 6/38

(52) U.S. Cl. PLT/157

(57) ABSTRACT

The new blueberry plant variety ‘FC14-062’ is provided. ‘FC14-062’ is a commercial variety intended for the hand harvest fresh fruit market. The variety is produced from a cross of ‘Blue Ribbon’ (female parent, patented Jul. 15, 2014) and ‘FC10-069’ (male parent, unpatented), which can be distinguished by its outstanding features.

### CROSS REFERENCE TO RELATED APPLICATIONS

None.

### STATEMENT REGARDING FEDERALLY-SPONSORED RESEARCH AND DEVELOPMENT

None.

Latin name of the genus and species: Family—Ericaceae. Genus—Vaccinium. Species—corymbosum hybrid.

Variety denomination: The new blueberry plant claimed is of the variety denominated ‘FC14-062’.

### BACKGROUND OF THE INVENTION

The present invention relates to the discovery of a new and distinct cultivar of northern highbush blueberry (Vaccinium corymbosum L. hybrid) plant, referred to as ‘FC14-062’, as herein described and illustrated. The new blueberry plant variety ‘FC14-062’ is a commercial variety intended for the fresh fruit market. The variety has high plant vigor, with an upright growth habit, and produces large fruit with excellent storability. ‘FC14-062’ was selected for its above average fruit quality and superior environmental adaptability compared to other early-mid-season varieties.

‘FC14-062’ differs from the female parent ‘Blue Ribbon’ in that ‘FC14-062’ has an upright plant shape and ‘Blue Ribbon’ has a sprawling plant shape. ‘FC14-062’ differs from the male parent ‘FC10-069’ in that it produces larger fruit with a better balance of leaves to fruit.

Pedigree and History: The new blueberry plant originated from a cross of ‘Blue Ribbon’ (female parent, patented Jul. 15, 2014) and ‘FC10-069’ (male parent, unpatented) made in Lowell, Oreg., USA in 2011.

The new blueberry variety ‘FC14-062’ was initially propagated in Lowell, Oreg., USA in 2011 and planted in the Spring of 2012.

‘FC14-062’ was selected for its superior fruit quality and plant performance. ‘FC14-062’ is a vigorous, precocious plant with concentrated fruit ripening and high yields competitive with other varieties in the same ripening season. The fruit is large with excellent storability to 45 days and beyond.

### SUMMARY OF THE INVENTION

The new blueberry plant variety ‘FC14-062’ has not been observed under all possible environmental conditions. The phenotype may vary somewhat with variations in environment and cultural practices such as temperature and light intensity without, however, any variance in genotype.

‘FC14-062’ is distinguished by its vigorous, strong growth habit and fruit quality. The following characteristics of the new cultivar have been repeatedly observed and are determined to be the unique characteristics of the new blueberry plant variety ‘FC14-062’:
1) Vigorous, strong plant growth with exceptional precocity
2) Early-mid season, concentrated ripening
3) Large fruit size with firm fruit texture
4) Very good, long-term cold storage performance

### BRIEF DESCRIPTION OF THE DRAWINGS

The accompanying photographic illustration shows typical specimens in full color of the foliage and fruit of the new variety ‘FC14-062.’ The colors are as nearly true as is reasonably possible in a color representation of this type.

FIG. 1 is a photograph of the new variety ‘FC14-062’, demonstrating the plant's leaves and growth habit in the fall. The plants were grown in Lowell, Oreg. and photographed on Nov. 16, 2018.

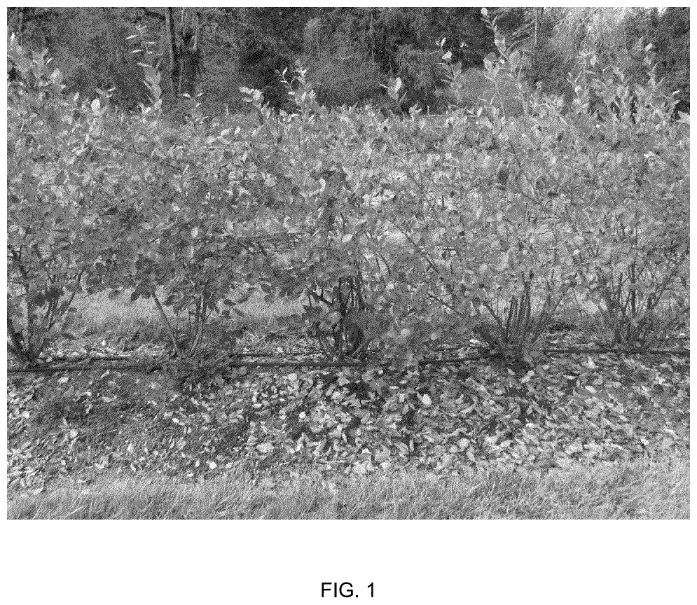

FIG. 2 is a photograph of the fruit cluster of the new variety ‘FC14-062’ grown in Lowell, Oreg. and photographed on Jun. 18, 2018.

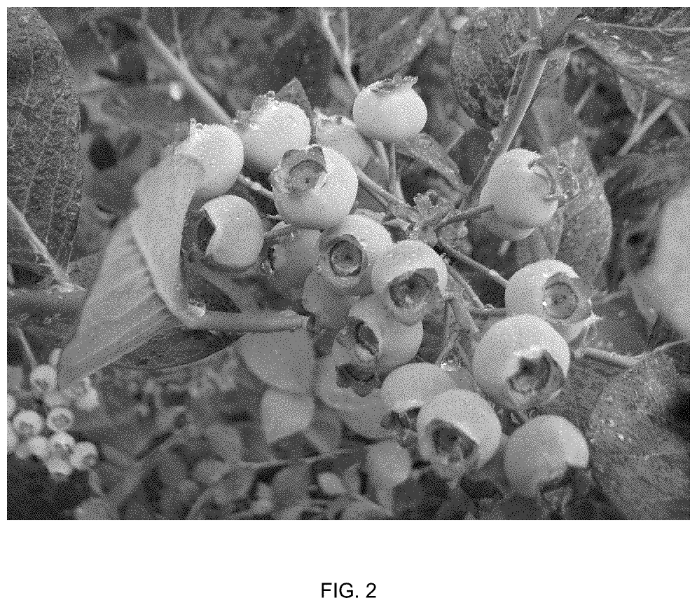

FIG. 3 is a photograph of the flowers of the new variety ‘FC14-062’ grown in Lowell, Oreg. and photographed on Apr. 18, 2018.

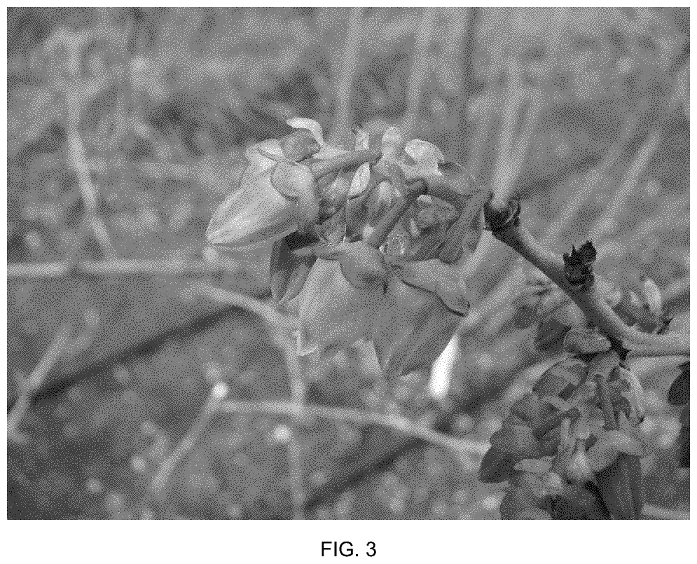

FIG. 4 is a photograph of the fall foliage of the new variety ‘FC14-062’ grown in Lowell, Oreg. and photographed on Nov. 16, 2018.

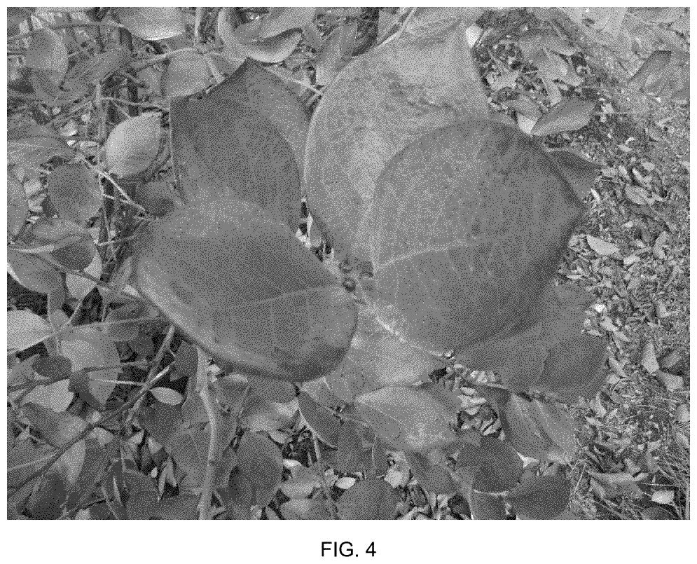

FIG. 5 is a photograph of early buds on the new variety ‘FC14-062’ grown in Lowell, Oreg. and photographed on Apr. 18, 2018.

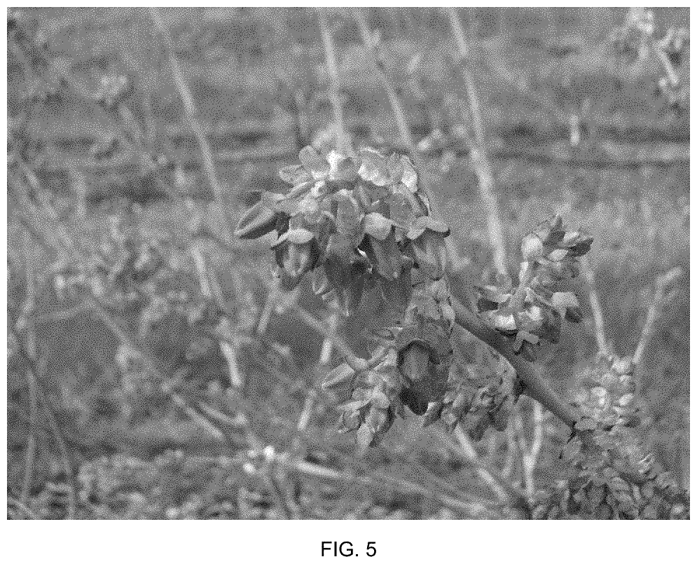

FIG. 6 is a photograph of one-year old canes on the new variety ‘FC14-062’ grown in Lowell, Oreg. and photographed on Apr. 18, 2018.

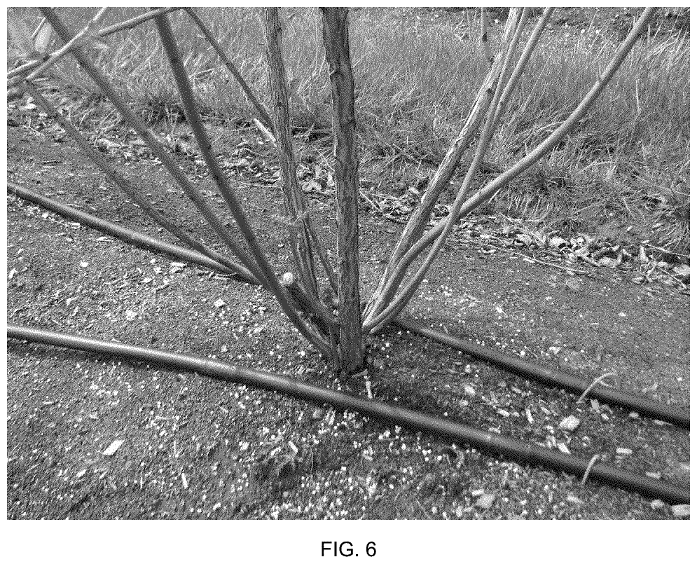

The colors in the photographs are as close as possible with the photographic and printing technology utilized. The color values cited in the detailed botanical description accurately describe the colors of the new blueberry.

### DETAILED BOTANICAL DESCRIPTION

The following detailed description sets forth the distinctive characteristics of ‘FC14-062’. The data which defines these characteristics was collected from asexual reproductions of the original selection. Dimensions, sizes, colors, and other characteristics are approximations and averages set forth as accurately as possible. The plant history was taken on plants approximately 3 to 4 years of age, and the descriptions relate to plants grown in the field located at 39252 Jasper-Lowell Road, Lowell, Oreg., USA. Descriptions of fruit characteristics were made on fruit grown at the field described above. Color designations are from “The Pantone Book of Color” (by Leatrice Eiseman and Lawrence Herbery, Harry N. Abrams, Inc., Publishers, New York 1990). Where the Pantone color designations differ from the colors of the photographs, the Pantone colors are accurate.
Classification:
   a. Family.—Ericaceae.
   b. Genus—Vaccinium.
   c. Species.—corymbosum.
   d. Common name.—Northern Highbush Blueberry.
Parentage: Female Parent ‘Blue Ribbon’ (patented Jul. 15, 2014). Male Parent ‘FC10-069’ (unpatented).
Market class: Commercial production of fresh fruit, but suitable for process market.
Plant:
   General.—a. Parentage — Female Parent — ‘Blue Ribbon’ (U.S. patent Jul. 15, 2014). Male Parent — ‘FC10-069’ (unpatented). b. Plant height — 137.668 cm. c. Plant width — 172.212 cm. d. Growth habit — Upright. e. Growth — High. f. Productivity — High. g. Cold hardiness — Good. h. Chilling requirement — Estimated minimum of 800-1000. i. Leafing — High. j. Twigginess — Somewhat twiggy. k. Resistance/susceptibility to root rot (Phytopthora cinnamomii) — Not overly susceptible. l. Resistance/susceptibility to stem blight (Botryosphaeria sp.) — Not overly susceptible. m. Resistance/susceptibility to Phomopsis Twig Blight (Phomopsis vaccinii) — Not overly susceptible. n. Resistance/susceptibility to Botrytis (Botrytis cinerea) — Not overly susceptible. o. Resistance/susceptibility to Leaf Spot (Septoria spp.) — Not overly susceptible. p. Resistance/susceptibility to Leaf Rust (Naohidemyces vaccinii) — Not overly susceptible. q. Resistance/susceptibility to bud mites (Acalatus vaccinii) — Not overly susceptible.
Stem:
   General.—a. Suckering tendency — Low. b. Mature cane color — Pantone color ‘Gravel’ 14-10114. c. Mature cane length — Average 142.758 cm. d. Mature cane width — Average 16.810 mm. e. Bark texture — Rough. f. Fall color on new shoots — Pantone color ‘Crimson’ 19-1762. g. Surface texture of new wood — Smooth. h. Internode length on strong, new shoots — 26.466 mm. i. Average number of buds per fruiting lateral — Average 7.2.
Foliage:
   General—a. Time of beginning of leaf bud burst — Mid April in Lowell, Oreg., Early April. b. Leaf color (Top side) — Pantone color ‘Chive’19-0323. c. Leaf color (Under side) — Pantone color ‘Sage’ 16-0421. d. Leaf arrangement — Alternate and Spirally: dextrose. e. Leaf shape — Broadly Elliptic. f. Leaf margins — Entire. g. Undulation of margin — None. h. Leaf venation — Anastomosing. i. Leaf apices — Slightly Acuminate. j. Leaf bases — Obtuse. k. Leaf Length — 68.212 mm. l. Leaf Width — 40.708 mm. m. Leaf length/width ratio — 1.67. n. Leaf nectarines — Absent. o. Pubescence of upper side — Absent. p. Pubescence of lower side — Absent. q. Cross sectional profile — Slightly Revolute. r. Longitudinal profile — Flat. s. Attitude — Porrect.
   Petioles.—a. Length — 3.628 mm. b. Width — Average 1.678 mm. c. Color — Pantone color ‘Hay’ 12-0418. d. Petiole surface texture — Smooth.
Flowers:
   General.—a. Time of beginning of flowering — Averaging mid to late April in Lowell, Oreg. b. Time of 50% anthesis — Averaging early May in Lowell, Oreg. c. Flower shape — Urceolate. d. Flower bud density — medium. e. Flower fragrance — None. f. Immature flower color — Pantone color ‘Geranium Pink’ 15-1922. g. Self-compatibility — Good. Approximately 72% of self-pollinated flowers set fruit in 2019.
   Corolla.—a. Color — Pantone color ‘Pastel Parchment’ 11-0603. b. Length — 9.322 mm. c. Width — 8.25 mm. d. Aperture width — 4.444 mm. e. Anthocyanin coloration of corolla — Low-None. f. Corolla ridges — Present. g. Protrusion of stigma — 0.0734 mm below the corolla.
   Inflorescence.—a. Length — 37.62 mm. b. Diameter — 39.154 mm. c. Length of peduncle — 19.224 mm. d. Surface texture of peduncle — Smooth. e. Color of peduncle — Pantone color ‘Holly berry’ 17-1633’. f. Length of pedicel — 8.902 mm. g. Surface texture of pedicel — Smooth. h. Color of pedicel — Pantone color ‘Holly berry’ 17-1633. i. Number of flowers per cluster — 7.4. j. Flower cluster density — Medium.
   Calyx (with sepals).—a. Diameter — 8.488 mm. b. Color (sepals) — Pantone color ‘Mellow Green’ 12-0426.
   Stamen.—a. Length — 6.46 mm. b. Number per flower — 10 stamen per flower. c. Filament color — Pantone color ‘Citron’ 12-0524 .
   Pistil.—a. Length — 9.63 mm. b. Ovary color (exterior) — Pantone color ‘Cypress’ 18-0322. c. Style length — 8.74 mm.
   Anther.—a. Length — 4.716 mm. b. Number — 2 per filament. c. Color — Pantone color ‘Buckskin’ 16-1342.
   Pollen.—a. Abundance — Moderate. b. Color — Pantone color ‘Cornhusk’ 12-0817.
Fruit:
   General.—a. Time of fruit ripening Averaging — July 12^{th} in Lowell, Oreg. b. Time of 50% maturity — Approximately July 12^{th} in Lowell, Oreg. c. Fruit development period — Averaging 72 days in Lowell, Oreg. d. Mean harvest date — Averaging July 16^{th}. e. Mean date last pick — Averaging August 1^{st}. f. Cluster density — Medium-high. g. Berries per cluster — 7.6 berries per cluster. h. Unripe fruit color — Pantone color ‘Apple Green’ 15-0543. i. Ripe Berry color — Pantone color ‘Purple Cloud’ 46-3919. j. Berry skin color after polishing — Pantone Color ‘Ebony’ 19-4104. k. Berry surface wax abundance — Medium. l. Berry flesh color — Pantone color ‘Water Lily’ 11-0304. m. Berry weight — Average 2.85 g. n. Berry Height from calyx to scar — 13.519 mm. o. Berry Diameter — 18.755 mm. p. Calyx aperture — Average 7.147 mm. q. Calyx depth — Average 1.075 mm. r. Pedicel length — Average 10.376 mm. s. Pedicel surface texture — Smooth. t. Berry detachment force — Medium-not difficult to pick, no scar tears. u. Berry shape — Oblate. v. Fruit stem scar — Small-medium, dry. w. Sweetness when ripe — Medium. x. Firmness when ripe — Firm. y. Acidity when ripe — Medium. z. Storage quality — Very Good.
Seed:
   General.—a. Seed abundance in fruit — Medium. b. Seed color — Pantone color ‘Brown Sugar’ 17-1134. c. Seed dry weight — 0.267 mg. per seed. d. Seed length — Average 1.5 mm.
Comparison between parental and commercial cultivars:

| Denomination | | State of | State of |
|---|---|---|---|
| of | Characteristic | expression of | expression of |
| similar | for | similar | candidate variety |
| variety | comparison | variety | ‘FC14-062’ |
| Draper | Vigor | Medium | High |
| Draper | Storability | Good | Excellent |
| Blue Ribbon | Yield | 7.2 lbs/plants | 9.7 lbs/plants |
| | | (3 yrs | (3 yrs |
| | | old) | old) |
| Blue Ribbon | Growth Habit | Sprawling | Upright |
| FC10-069 | Fruit size | Medium | Large |

1. A new and distinct variety of blueberry plant named ‘FC14-062’, substantially as illustrated and described herein.
